(19) United States
(12) Patent Application Publication
(10) Pub. No.: US 2009/0270968 A1
(43) Pub. Date: Oct. 29, 2009

(54) Stent for Branched Vessel and Balloon Catheter Unit having the Same

(76) Inventor: Choong Won Go, Seoul (KR)

Correspondence Address: PARK LAW FIRM, 3255 WILSHIRE BLVD, SUITE 1110, LOS ANGELES, CA, 90010, US

(21) Appl. No.: 12/303,514

(22) PCT Filed: Jun. 5, 2007
(86) PCT No.: PCT/KR2007/002719

§ 371 Date: Dec. 4, 2008

(30) Foreign Application Priority Data
Jun. 7, 2006 (KR) 10-2006-0051086

(51) Int. Cl. A61F 2/06; A61M 29/00

(52) U.S. Cl. 623/1.11; 623/1.15; 604/96.01

(57) ABSTRACT

A stent for branched vessel by which a stent for main vessel can be inserted in the main vessel easily without interference with the stent for branched vessel that was already installed is disclosed. The stent for branched vessel of the present invention for expanding a constricted branched vessel is characterized in that a plurality of wave-form modules make a net shape so that a connectionless slit is formed lengthwise at the one end portion of the stent.

### TECHNICAL FIELD

The present invention relates to a stent for branched vessel and a balloon catheter unit having the same, and more specifically to a stent for branched vessel by which the stent for main vessel can be easily inserted in the main vessel without interference with the stent for branched vessel that was already installed, and also a balloon catheter unit by which it is possible to exactly place the stent for branched vessel at the required position of the branched vessel.

### BACKGROUND ART

A stent is an implant inserted and installed in the human vessel. Fitted on a balloon catheter and guided by a guide steel wire, it plays the role of dilating the constricted vessel and reinforcing the vessel wall as the diameter is dilated by the expansion of the balloon catheter after it reaches the constricted vessel.

A general method of operation using stent will be briefly described referring to FIG. 1 and FIG. 2. A guide steel wire 11 is inserted in the constricted part of the vessel that needs treatment, and the balloon catheter is advanced along the guide steel wire 11 to let it reach the constricted part. Here, the balloon catheter comprises a guide steel wire tube 12 disposed on the periphery of the guide steel wire 11, a balloon 13 which is installed on the outside of the guide steel wire tube 12 and expands when liquid is supplied, a liquid supply tube 14 which is disposed on the outer circumference of the guide steel wire so as to supply liquid to the balloon 13, and a stent 15 which is fitted on the outer circumference of the balloon and expands the constricted vessel as the diameter is dilated by the expansion of the balloon. Accordingly, as liquid is injected into the balloon 13 through the liquid supply tube 14 to expand the balloon, the diameter of the stent 15 is dilated to expand the constricted vessel. Later, when liquid is discharged from the balloon, only the stent 15 remains installed in the vessel, and the guide steel wire tube 12 is retreated along the guide steel wire 11 to be withdrawn out of the human body. Next, when the guide steel wire is finally retreated, the operation is completed.

Here, the stent 15 is a net shape in which a plurality of wave-form modules are connected in a ring form, as shown in FIG. 3, or could be a net shape in which a plurality of wave-form modules are connected through connecting members 15-1, as shown in FIG. 4. Namely, the stents are developed and used in various net shapes as necessary.

However, when the stent 15 configured as described above is fitted on a balloon catheter for surgical operation, the following problem arises. If operation is required because constriction has occurred near the vessel branch point where the branched vessel is branched from the main vessel, there may be a case that the stent for the branched vessel that was already installed in the branched vessel is protruded into the main vessel and blocks part of the main vessel, in this situation the balloon catheter for the main vessel needs to advance through the hole of the stent for the branched vessel to be placed in the main vessel. But the problem is that it is difficult to place the balloon catheter for the main vessel at the required position of the main vessel due to interference with the stent for the branched vessel.

### DISCLOSURE OF INVENTION

### Technical Problem

Accordingly, it is an object of the present invention to provide a stent for branched vessel by which a stent for main vessel can be installed easily in the main vessel without interference with the stent for branched vessel that was already installed. It is another object of the present invention to provide a balloon catheter by which it is possible to place a stent for branched vessel exactly at the required position of the branched vessel.

### Technical Solution

To achieve the above objects, there is provided a stent for branched vessel for expanding a constricted branched vessel, wherein a plurality of wave-form modules make a net shape so that a connectionless slit is formed lengthwise at the one end portion of said stent.

### ADVANTAGEOUS EFFECTS

According to the present invention, it is possible to install the stent for main vessel easily without interference with the stent for branched vessel that was already installed, as a connectionless slit or a connectionless slot is dilated and opened. Also, by the balloon catheter unit including the stent for branched vessel, it is possible to place the stent for branched vessel exactly at the required position of the branched vessel.

### BRIEF DESCRIPTION OF THE DRAWINGS

These and other features, aspects, and advantages of preferred embodiments of the present invention will be more fully described in the following detailed description, taken in conjunction with the accompanying drawings. In the drawings:

FIG. 1 and FIG. 2 are schematic views for describing the operation method using a commonly used stent;

FIGS. 3 and 4 are lateral views showing a conventional ordinary stent;

FIG. 5 is a perspective view of a stent for branched vessel according to an embodiment of the present invention;

FIG. 6 is a lateral view of the stent for branched vessel according to the present invention;

FIGS. 7 to 12 are schematic views for describing the operation method and its action of the stent for branched vessel according to FIG. 5 and FIG. 6;

FIG. 13 is a perspective view of a stent for branched vessel according to another embodiment of the present invention;

FIG. 14 is a lateral view of the stent for branched vessel according to another embodiment of the present invention;

FIGS. 15 through 20 are schematic views for describing the operation method and its action of a stent for branched vessel according to FIG. 13 and FIG. 14;

FIG. 21 is an exploded perspective view of the expanded state of a balloon catheter unit for branched vessel according to the present invention;

FIG. 22 is an assembled perspective view of FIG. 21;

FIGS. 23 to 25 are schematic views for describing the operation method and its action of a balloon catheter unit according to FIG. 21;

FIG. 26 is an exploded perspective view of the expanded state of a balloon catheter unit for branched vessel according to another embodiment of the present invention; and

FIG. 27 is an assembled perspective view of FIG. 26.

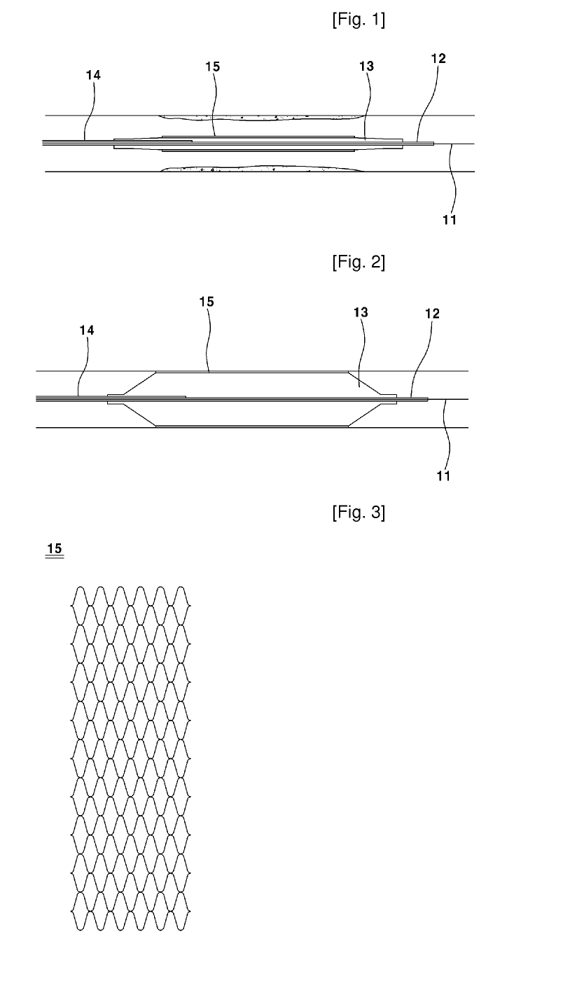

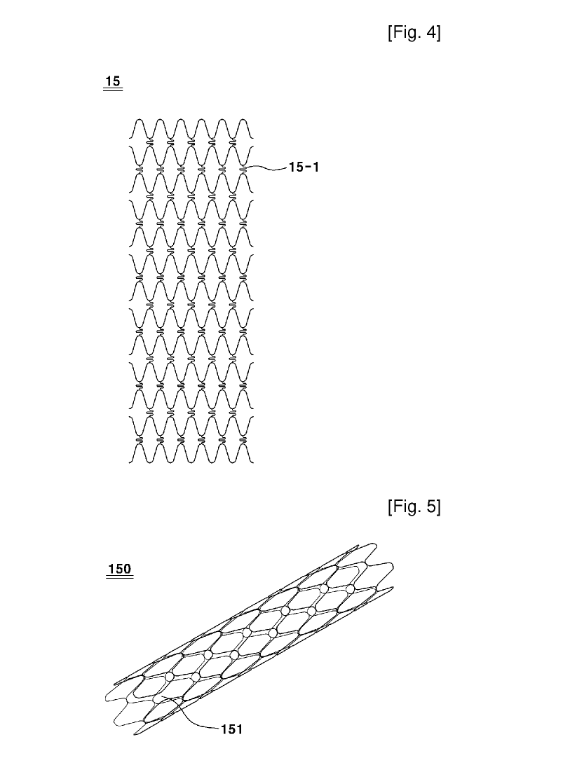

### BEST MODE FOR CARRYING OUT THE INVENTION

Below will be described in detail a stent for branched vessel according to the present invention and a balloon catheter unit having the same with reference to the accompanying drawings.

(Stent for Branched Vessel)

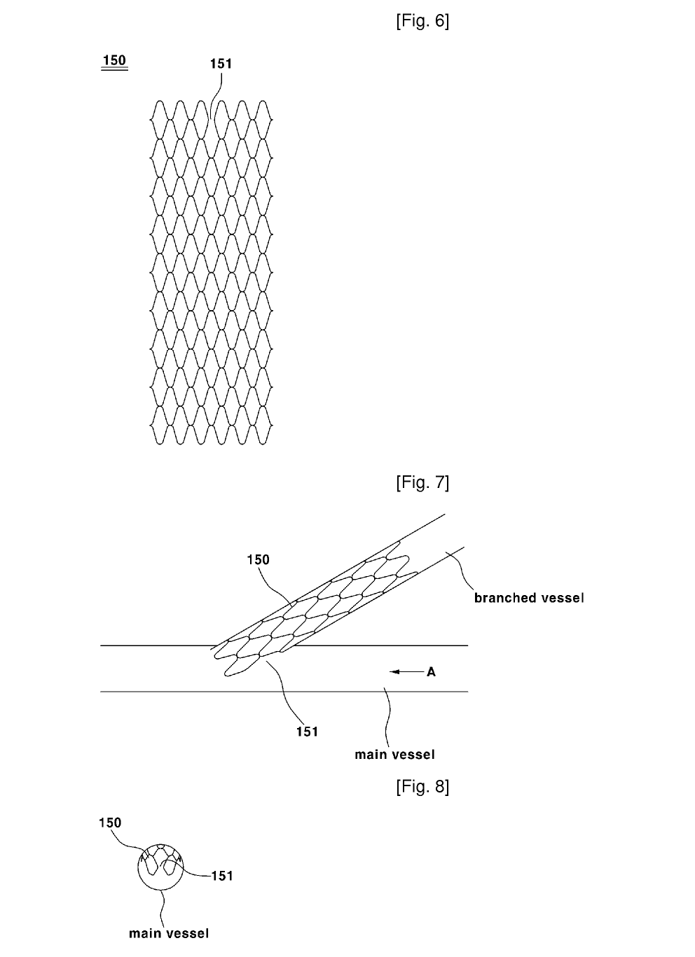

FIG. 5 is a perspective view of a stent for branched vessel according to an embodiment of the present invention, and FIG. 6 is a lateral view of the stent for branched vessel according to the present invention.

Referring to these drawings, a stent 150 for branched vessel according to the present invention is to expand a constricted branched vessel, characterized in that a plurality of wave-form modules make a net shape in one end portion of the stent in such a way that connectionless slit 151 is formed lengthwise.

The stent 150 for branched vessel, which will be described later, is to expand the branched vessel with the diameter dilated by the expansion of a balloon, after it is fitted on the periphery of the balloon catheter disposed on the periphery of the branched vessel guide steel wire put into the branched vessel branched from the main vessel and reaches the branched vessel. The material of the stent is not especially limited, but it is made of stainless steel or cobalt alloy, and it may be coated with chemicals to prevent re-constriction.

The stent 150 for branched vessel is, as shown in FIG. 5 and FIG. 6, can be of a net shape in which a plurality of wave-form modules are connected in a ring form, or although it is not shown, it can be of course modified in various net shapes in public domain, in which a plurality of wave-form modules are connected by connecting members.

Here, the stent 150 according to the present invention is characterized in that a plurality of wave-form modules make a net shape in such a way that a connectionless slit 151 is formed lengthwise at one end portion of the stent. As mentioned in the prior art, if constriction occurred near the vessel branch point where the branched vessel is branched from the main vessel and a stent for branched vessel is installed, this stent for branched vessel is protruded into the main vessel, so interference occurs later when a stent for main vessel is to be installed.

But the stent 150 for branched vessel according the present invention is placed in a branched vessel in such a way that the part where the connectionless slit 151 is formed is protruded into the main vessel, and then the slit is dilated and opened in such way as will be described later. Therefore, when a stent for main vessel is installed, interference with the stent for branched vessel is minimized, so it is possible to install a stent for main vessel easily in the main vessel.

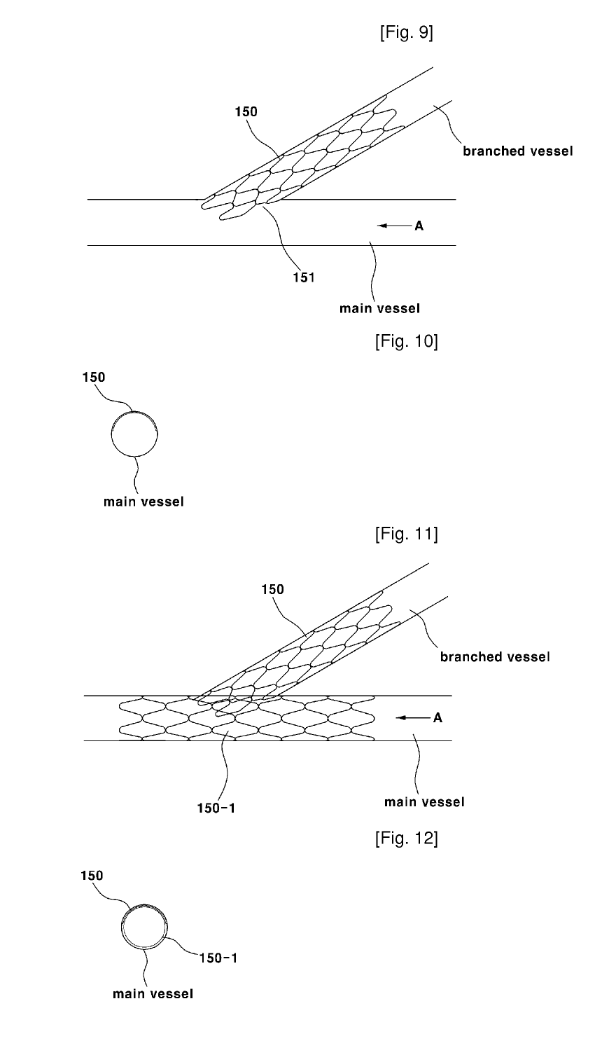

Referring to FIGS. 7 to 12, the operation method and its action of the stent for branched vessel according to the present invention will be described. FIG. 7 is a schematic view showing a state of a branched vessel expanded by the stent for branched vessel according to the present invention; FIG. 8 is a cross sectional view of FIG. 7 as seen from direction A; FIG. 9 is a schematic view showing a state in which the connectionless slit of the stent for branched vessel according to the present invention is dilated and opened; FIG. 10 is a cross sectional view of FIG. 9 as seen from direction A; FIG. 11 is a schematic view showing the state in which a stent for main vessel is expanded into the dilated and opened connectionless slit of the stent for branched vessel according to the present invention; and FIG. 12 is a cross sectional view of FIG. 11 as seen from direction A.

Referring to these drawings, first as shown in FIG. 7 and FIG. 8, the stent 150 for branched vessel according to the present invention is placed in the branched vessel in such a way that the part where a connectionless slit 151 is formed is protruded into the main vessel, and then the diameter of the stent is dilated to expand the branched vessel. After that, as shown in FIG. 9 and FIG. 10, the connectionless slit 151 of the stent for branched vessel protruded into the main vessel is dilated and opened. Here, the dilation and opening method of the connectionless slit is described. As an example, after the balloon catheter is passed through the slit, liquid is injected. Then, as the balloon is expanded to push out the slit to both sides, the slit is dilated and opened. Like this, after the connectionless slit 151 of the stent for branched vessel is dilated and opened, a stent for main vessel is put into the dilated and opened space and expanded, and then the stent for main vessel expands the main vessel. At this time, because the connectionless slit 151 is dilated and opened, the stent 150-1 for branched vessel can be placed at the exact position in the main vessel without interference with the stent 150 for branched vessel.

As described above, the stent 150 for branched vessel according to the present invention does not interfere with the entry of a main vessel stent (more specifically, a balloon catheter for main vessel fitted with a main vessel stent), because the connectionless slit 151 protruded into the main vessel is dilated and expanded. Because of that, the main vessel stent 150-1 is placed at the exact position, so it has an advantage that the degree of operation satisfaction can be improved.

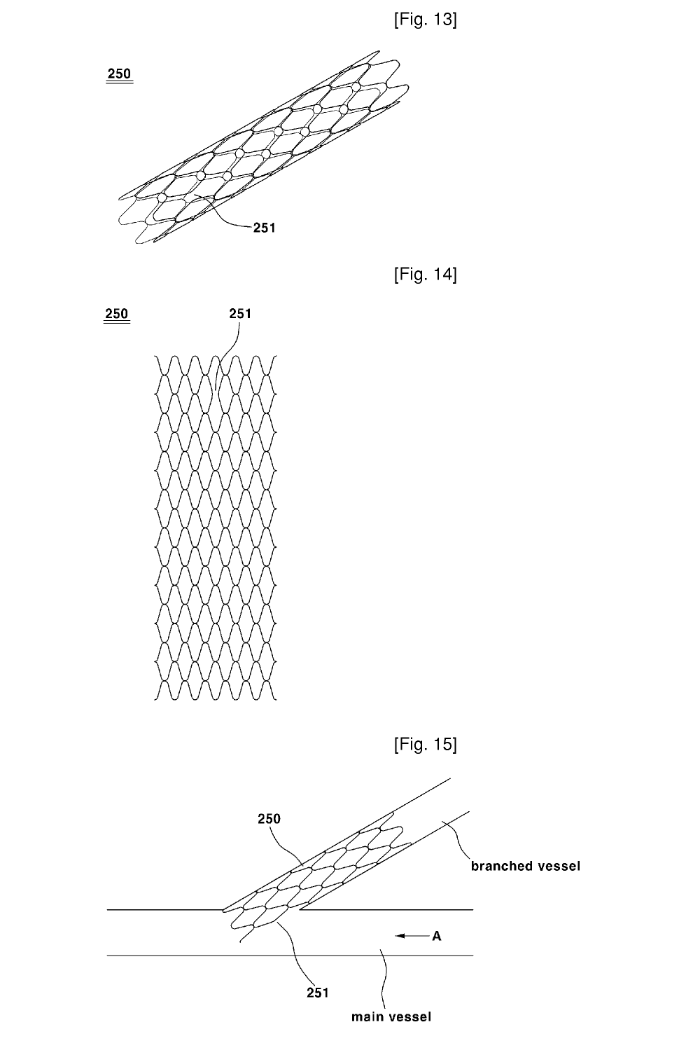

FIG. 13 is a perspective view of a stent for branched vessel according to another embodiment of the present invention, and FIG. 14 is a lateral view of the stent for branched vessel according to another embodiment of the present invention. The present embodiment is characterized in that a plurality of wave-form modules make a net shape in such a way that a connectionless slot 251 is formed lengthwise on the flank of one end of a stent 250 for branched vessel. In the previous embodiment, because the slit 151 is formed in the stent, the one end portion of the stent is opened, but in the present embodiment, a connectionless slot 251 is formed on one side of the stent, so the one end portion is not opened.

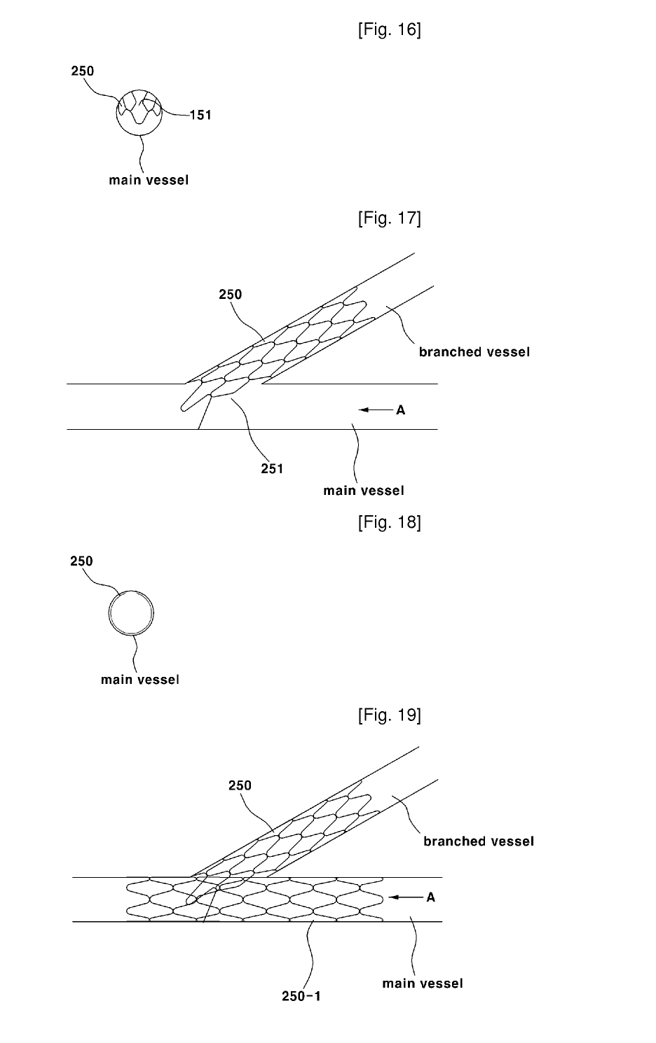

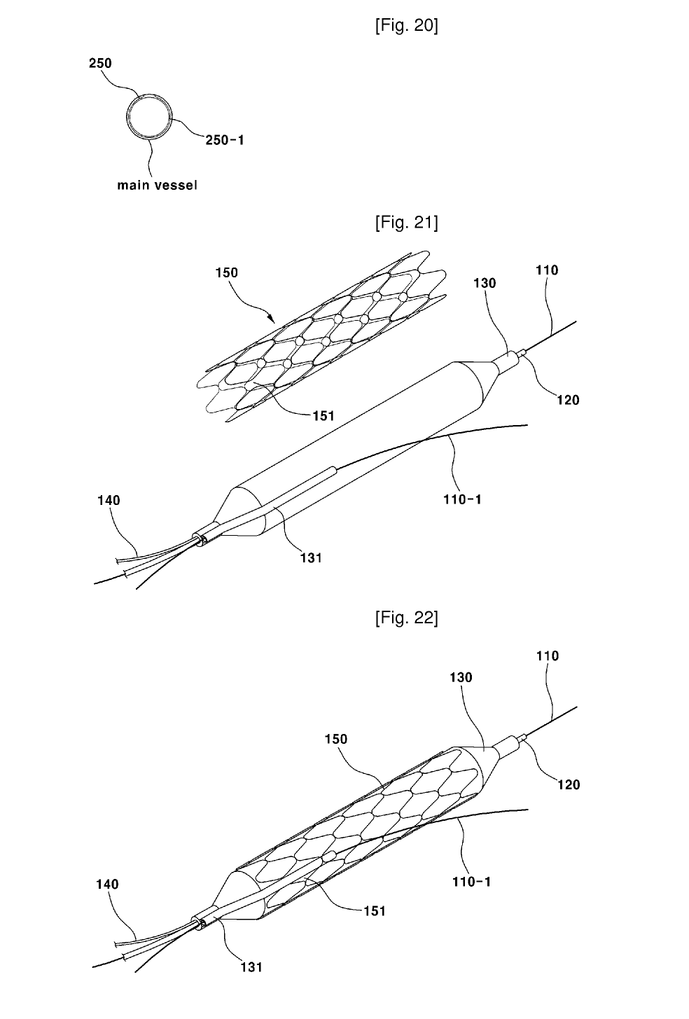

Since the method of using the stent 250 is substantially the same with the previous embodiment, it will be described briefly. First, as shown in FIG. 15 and FIG. 16, the stent for branched vessel according to the present embodiment is placed in a branched vessel in such a way that the part where the connectionless slot 251 is formed is protruded into the main vessel, and then the diameter is dilated to expand the branched vessel. After that, as shown in FIG. 17, the connectionless slot 251 of the stent for branched vessel protruded into the main vessel is enlarged. The enlarged connectionless slot 251 is opened circularly as shown in FIG. 18. While the connectionless slot 251 of the stent for branched vessel is dilated and opened like this, a stent 250-1 for main vessel is put through the dilated and opened space and expanded, and then the stent for main vessel expands the main vessel, as shown if FIGS. 19 and 20. Accordingly, because the connectionless slot 251 is dilated and opened, it is possible to place the stent for main vessel at the exact position in the main vessel without interference with the stent for branched vessel. Here, FIGS. 16, 18 and 20 are cross sectional views of FIGS. 15, 17 and 19, respectively, as seen from direction A.

### MODE FOR THE INVENTION

### Balloon Catheter Unit for Branched Vessel

Below will be described a balloon catheter unit for branched vessel according to the present invention. The balloon catheter unit for branched vessel of the present invention comprises a balloon catheter for branched vessel and stents 150 and 250. This balloon catheter unit requires skill of positioning the connectionless slit 151 that was already described or the initial point of the connectionless slot 251, at the vessel branch point.

FIG. 21 is an exploded perspective view showing the expanded state of a balloon catheter unit for branched vessel according to one embodiment of the present invention, and FIG. 22 is an assembled perspective view of FIG. 21. Referring to these drawings, the balloon catheter unit of the present embodiment comprises a branched vessel guide steel wire tube 120, a balloon 130, a liquid supply tube 140 and a stent 150 for branched vessel.

The branched vessel guide steel wire tube 120 is disposed on the periphery of the branched vessel guide steel wire 110 that was put into the branched vessel branched from the main vessel, and is advanced into the branched vessel guided by the branched vessel guide steel wire 110. The balloon 130 is installed on the periphery of the branched vessel guide steel wire tube 120, and when liquid is supplied to the inside it expands and when liquid is discharged it contracts. And, at the end near the branch of the balloon is attached and installed a main vessel guide steel wire tube 131 that is disposed on the periphery of the main vessel guide steel wire 110-1 put in along the main vessel. The liquid supply tube 140 is disposed on the flank of the branched vessel guide steel wire 110 so as to supply liquid to the balloon 130, and then is inserted into the balloon 130. In the stent for branched vessel 150, as mentioned above, a plurality of wave-form modules make a net shape in such a way that the connectionless slit 151 is formed lengthwise at the one end portion the stent. The stent is fitted on the periphery of the balloon so that the connectionless slit 151 is disposed on the main vessel guide steel wire tube of the balloon.

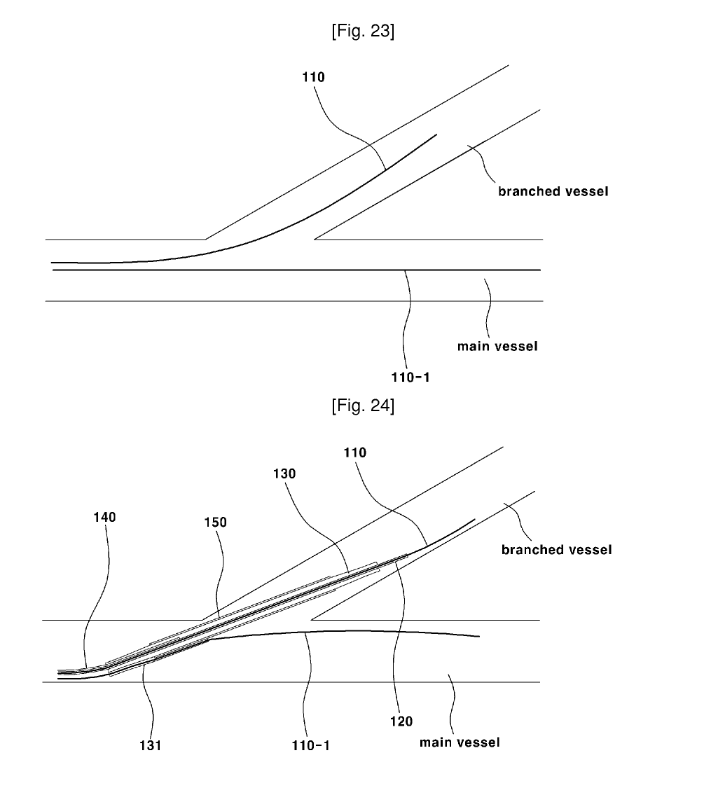

A balloon catheter unit for branched vessel according to the present invention having such a configuration can be positioned in such a way that the connectionless slit portion of the stent for branched vessel is protruded into the main vessel, and this will be described with reference to FIGS. 23 to 25.

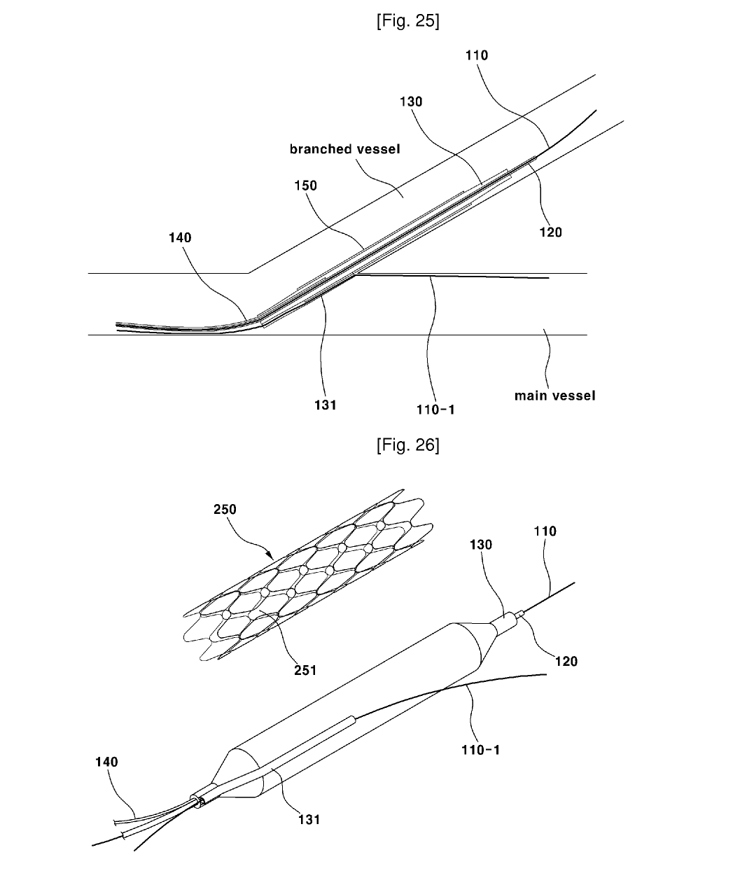

First, as shown in FIG. 23, a branched vessel guide steel wire 110 is put into the branched vessel branched from the main vessel, and a main vessel guide steel wire 110-1 is put into the main vessel. After that, as shown in FIG. 24, with the main vessel guide steel wire tube 131 disposed on the main vessel guide steel wire 110-1, the balloon catheter unit is advanced along the branched vessel guide steel wire 110. As the balloon catheter unit advances, as shown in FIG. 25, the main vessel guide steel wire 110-1 is bent at the vessel branch point and has the end portion of the main vessel guide steel wire 110-1 caught by the vessel branch point, so the advance of the balloon catheter unit is stopped. By the stopped balloon catheter unit, the connectionless slit 151 portion of the stent for branched vessel maintains a state protruded into the main vessel. Namely, only the connectionless slit 151 portion of the stent for branched vessel protrudes into the main vessel.

From then on liquid is supplied to the balloon 130 through the liquid supply tube 140 to expand the balloon 130 and the stent 150 for branched vessel. When the liquid is discharged from the balloon, the balloon 130 is contracted so only the stent 150 for branched vessel keeps the branched vessel expanded. When the balloon catheter for branched vessel is retreated later, it becomes just like that depicted in FIG. 7 and FIG. 8. After that, the balloon is inserted along the main vessel guide steel wire 110-1 to dilate and open the slit 151 of the stent for branched vessel, such that it becomes like that depicted in FIG. 9 and FIG. 10. Next, when the balloon catheter fitted with a stent for main vessel is moved along the main vessel guide steel wire and then expanded, the implant operation is completed as shown in FIG. 11 and FIG. 12.

As described above, because the balloon catheter unit for branched vessel according to the present invention can position the connectionless slit 151 portion of the stent 150 for branched vessel in the main vessel with reference to the vessel branch point, later it is possible to dilate and open the slit of the stent for branched vessel exactly without error. Accordingly, it has an advantage that the stent for main vessel can be installed in the main vessel without interference with the stent for branched vessel.

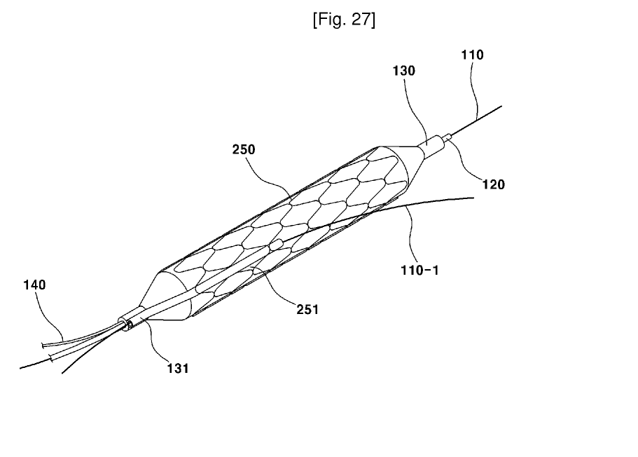

Meanwhile, FIG. 26 is an exploded perspective view showing the expanded state of a balloon catheter unit for branched vessel according to another embodiment of the present invention, and FIG. 27 is an assembled perspective view of FIG. 26. Referring to these drawings, the balloon catheter unit of the present embodiment is characterized by comprising a branched vessel guide steel wire tube 120 which is disposed on the periphery of a branched vessel guide wire 110 put into the branched vessel branched from the main vessel; a balloon 130 which is installed on the periphery of the branched vessel guide steel tube 120 and expands when liquid is supplied, and in which a main vessel guide steel wire tube 131 disposed on the periphery of the main vessel guide steel wire put into the main vessel is attached and installed near the end of the branch; a liquid supply tube 140 which is disposed on the flank of the branched vessel guide steel wire 131 so as to supply liquid to the balloon 130; and a stent 250 for branched vessel in which a plurality of wave-modules make a net shape so that a connectionless slot 251 is formed lengthwise on the flank of one end, and which is fitted on the periphery of the balloon so that the connectionless slot is disposed in the main vessel guide steel wire tube 131 of the balloon.

Compared with the previous embodiments, the above-described embodiment has a connectionless slot 251 formed in the stent for branched vessel, but the action is substantially the same, so specific description is omitted below.

The present invention has been illustrated and described in connection with specific embodiments, but those skilled in the art should easily know that diverse alterations and modifications are possible within the limit of not departing from the idea and domain of the invention shown by the scope of the following claims. As an example, so far only that the stent for branched vessel is expanded first in the branched vessel and the stent for main vessel is expanded later has been described, but it is obvious that it is possible to expand first the main vessel stent in the main vessel and expand later the stent for branched vessel in the branched vessel.

### INDUSTRIAL APPLICABILITY

As described above, by the stent for branched vessel according to the present invention, it is possible to install the stent for main vessel easily without interference with the stent for branched vessel that was already installed, as a connectionless slit or a connectionless slot is dilated and opened. Also, by the balloon catheter unit including the stent for branched vessel, it is possible to place the stent for branched vessel exactly at the required position of the branched vessel.

1. A stent for branched vessel for expanding a constricted branched vessel, wherein a plurality of wave-form modules make a net shape so that a connectionless slit is formed lengthwise at the one end portion of said stent.

2. A stent for branched vessel for expanding a constricted branched vessel, wherein a plurality of wave-form modules make a net shape so that a connectionless slot is formed lengthwise on the flank of one end of said stent.

3. A balloon catheter unit for branched vessel comprising:
a branched vessel guide steel wire tube which is disposed on the periphery of a branched vessel guide wire put into the branched vessel branched from the main vessel;
a balloon which is installed on the periphery of said branched vessel guide steel tube and is expanded by liquid supply, and in which a main vessel guide steel wire tube disposed on the periphery of the main vessel guide steel wire put into the main vessel is attached and installed near the end of the branch;
a liquid supply tube which is disposed on the flank of the branched vessel guide steel wire so as to supply liquid to said balloon; and
a stent for branched vessel in which a plurality of wave-modules make a net shape so that a connectionless slit is formed lengthwise at the one end portion of the stent, and which is fitted on the periphery of the balloon so that said connectionless slit is disposed in the main vessel guide steel wire tube of the balloon.

4. A balloon catheter unit for branched vessel comprising:
a branched vessel guide steel wire tube which is disposed on the periphery of a branched vessel guide wire put into the branched vessel branched from the main vessel;
a balloon which is installed on the periphery of said branched vessel guide steel tube and is expanded by liquid supply, and in which a main vessel guide steel wire tube disposed on the periphery of the main vessel guide steel wire put into the main vessel is attached and installed near the end of the branch;
a liquid supply tube which is disposed on the flank of the branched vessel guide steel wire so as to supply liquid to said balloon; and
a stent for branched vessel in which a plurality of wave-modules make a net shape so that a connectionless slot is formed lengthwise on the flank of one end, and which is fitted on the periphery of the balloon so that said connectionless slot is disposed in the main vessel guide steel wire tube of the balloon.
